(19) United States
(12) Patent Application Publication
(10) Pub. No.: US 2011/0124590 A1
(43) Pub. Date: May 26, 2011

(54) Oil-in-Water Emulsion Comprising Solvent, Water, Surfactant and Pesticide

(75) Inventors: Christian Sowa, Neustadt (DE); Tatjana Levy, Mannheim (DE); Ralf Vogel, Limburgerhof (DE); Wolfgang Meier, Limburgerhof (DE); Michael Krapp, Altrip (DE); Jurith Montag, Limburgerhof (DE); Claude Taranta, Stutensee (DE)

(73) Assignee: BASF SE, Ludwigshafen (DE)

(21) Appl. No.: 13/055,015

(22) PCT Filed: Jul. 14, 2009
(86) PCT No.: PCT/EP09/58983

§ 371 Date: Jan. 20, 2011

(30) Foreign Application Priority Data
Jul. 24, 2008 (EP) 08161046.1

(51) Int. Cl. A01N 43/04; A01N 43/54; A01N 43/40; A61K 31/417; A01N 43/16; A01P 3/00; A01P 1/00

(52) U.S. Cl. 514/37; 514/269; 514/355; 514/399; 514/53

(57) ABSTRACT

The present invention relates to an oil-in-water emulsion comprising solvent (A), water (B), surfactant (C) and at least 2% by weight of pesticide (D) based on the emulsion, wherein the solvent comprises an aromatic hydrocarbon (a) and a ketone (b). Furthermore, the invention relates to a process for the preparation of an oil-in-water emulsion by mixing solvent (A), water (B), surfactant (C) and at least 2% by weight of pesticide (D) based on the emulsion with one another. Further subject matter are an agrochemical formulation comprising the emulsion according to the invention and further adjuvants; a method of controlling phytopathogenic fungi and/or undesired vegetation and/or undesired attack by insects or mites and/or for regulating the growth of plants; a method of controlling undesired attack by insects or mites on plants and/or of controlling phytopathogenic fungi and/or of controlling undesired vegetation; and seed treated with an agrochemical formulation according to the invention.

The present invention relates to an oil-in-water emulsion comprising solvent (A), water (B), surfactant (C) and at least 2% by weight of pesticide (D) based on the emulsion. Furthermore, the invention relates to a process for the preparation of an oil-in-water emulsion by mixing solvent (A), water (B), surfactant (C) and at least 2% by weight of pesticide (D) based on the emulsion with one another. Further subject matter are an agrochemical formulation comprising the emulsion according to the invention and further adjuvants; a method of controlling phytopathogenic fungi and/or undesired vegetation and/or undesired attack by insects or mites and/or for regulating the growth of plants; a method of controlling undesired attack by insects or mites on plants and/or of controlling phytopathogenic fungi and/or of controlling undesired vegetation; and seed treated with an agrochemical formulation according to the invention. The present invention comprises combinations of preferred features with other preferred features.

Oil-in-water emulsions comprising pesticide, solvent, water and surfactant are generally known:

WO 2002/43488 discloses oil-in-water emulsions comprising insecticide, water, solvent, an anionic surfactant, two nonionic surfactants and film former/thickener. Suitable solvents are esters of aromatic mono- and diacids. Cosolvents which are mentioned are, inter alia, ketones such as 2-heptanone.

WO 2007/057028 discloses oil-in-water emulsions for plant protection comprising avermectins, esters of fatty acids as solvent, emulsifiers, water and one or more cosolvents which have a solubility in water of less than 10% at 25° C. Examples of cosolvents which are mentioned are aromatic hydrocarbons or cyclic aliphatic ketones.

DE 69012487 discloses oil-in-water emulsions comprising a pyrethroid, a film former and surfactant. Suitable solvents are solvent pairs of aromatic hydrocarbon and phthalic acid derivatives.

WO 2007/017501 discloses an emulsion concentrate (EC) comprising a phenylsemi-carbazone and a solvent system comprising butyrolactone, aliphatic and/or aromatic ketone and optionally an aromatic hydrocarbon. For use against insects, the EC formulation is usually diluted with water in order to obtain the spray solution.

It was an object of the present invention to develop a liquid agrochemical formulation which is highly storage-stable and permits the addition of large amounts of fat-soluble and water-soluble adjuvants. It was a further object to find a liquid agrochemical formulation which has a higher biological activity than other liquid formulations of the same active substance.

This object was achieved by an oil-in-water emulsion comprising solvent, water, surfactant and at least 2% by weight of pesticide based on the emulsion, wherein the solvent comprises an aromatic hydrocarbon and a ketone.

An oil-in-water emulsion (EW) is a conventional type of formulation in the field of the agrochemical formulations. In EW formulations of a pesticide, the emulsion is preformed in the commercial product and is conventionally diluted with a carrier, such as water, when making up the spray mixture. They therefore differ from emulsion concentrates (EC) which comprise pesticides in an anhydrous formulation in organic solvents. EC formulations form an emulsion only when making up the spray mixture, which emulsion will then comprise the pesticide only in small amounts of less than 0.5% by weight, based on the emulsion. The disadvantage of EC formulations is the high content of organic solvents.

The oil-in-water emulsion according to the invention comprises an aqueous phase as the continuous phase and an oil phase as the disperse phase. The oil phase usually forms droplets within the aqueous phase. The mean droplet size is usually in the region of at least 0.1 μm, preferably at least 0.3 μm, in particular at least 0.5 μm. It is preferably in the range of from 0.1 to 1.2 μm, in particular of from 0.3 to 0.9 μm and specifically of from 0.5 to 0.8 μm. The mean droplet size can be determined by particle-size measurement by means of laser diffraction, for example using a Malvern Mastersizer 2000.

The emulsion according to the invention can comprise at least 5% by weight, preferably at least 10% by weight and especially preferably at least 15% by weight of water based on the total weight of the emulsion. The emulsion according to the invention can comprise from 5 to 70% by weight, preferably from 10 to 60% by weight and especially preferably from 15 to 50% by weight of water, based on the total weight of the emulsion.

The solvent (A) of the emulsion according to the invention comprises an aromatic hydrocarbon (a) and a ketone (b). Preferably, the solvent comprises a further ketone (c). It is especially preferred that the ketone (b) is an aliphatic ketone, and the ketone (c) is an aromatic ketone. In particular, the aromatic hydrocarbon (a) has a boiling point of at least 160° C., the ketone (b) is an aliphatic ketone having 6 to 12 C atoms, and the ketone (c) is acetophenone or its derivatives. It is specifically preferred that the aromatic hydrocarbon (a) is an aromatic hydrocarbon mixture with a boiling point of at least 160° C., the ketone (b) is 2-heptanone and the ketone (c) is acetophenone.

Aromatic hydrocarbons (a) are compounds which consist of carbon and hydrogen and which comprise aromatic groups. Preferred are aromatic hydrocarbons or their mixtures with an initial boiling point of at least 160° C., preferably at least 180° C.

Examples of aromatic hydrocarbons are benzene, toluene, o-, m- or p-xylene, naphthalene, biphenyl, o- or m-terphenyl, aromatic hydrocarbons which are mono- or polysubstituted by C1-C20-alkyl, such as ethylbenzene, dodecylbenzene, tetradecyl-benzene, hexadecylbenzene, methylnaphthalene, diisopropylnaphthalene, hexyl-naphthalene or decylnaphthalene. Others which are suitable are aromatic hydrocarbon mixtures with an initial boiling point of at least 160° C. Such compounds are for example commercially available from ExxonMobil or BP under the following trade names: Solvesso® 100, Solvesso® 150, Solvesso® 200, Solvesso® 150ND, Solvesso® 200ND, Aromatic® 150, Aromatic® 200, Hydrosol® A 200, Hydrosol® A 230/270, Caromax® 20, Caromax® 28, Aromat® K 150, Aromat® K 200, Shellsol® A 150, Shellsol0 A 100, Fin® FAS-TX 150, Fin® FAS-TX 200. Preferred aromatic hydrocarbons are aromatic hydrocarbon mixtures with an initial boiling point of at least 160° C., preferably at least 180° C. Mixtures of the above aromatic hydrocarbons are also possible. Usually, the emulsion according to the invention comprises from 1 to 50% by weight, preferably from 5 to 40% by weight, especially preferably from 10 to 30% by weight of aromatic hydrocarbon, in each case based on the total weight of the emulsion.

Ketones (b) or further ketones (c) are compounds which comprise at least one ketone group and which are present in liquid form at 20° C. They preferably consist of carbon, hydrogen and oxygen. In particular, they do not comprise any further functional groups besides at least one ketone group. Preferred ketones are those with a boiling point of at least 60° C., preferably at least 80° C. and in particular at least 100° C. Both aliphatic and aromatic ketones are suitable. Usually, the emulsion according to the invention comprises from 1 to 70% by weight, preferably from 5 to 50% by weight, especially preferably from 10 to 40% by weight of ketone, in each case based on the total weight of the emulsion.

Suitable aliphatic ketones usually comprise from 5 to 18, preferably from 6 to 14, in particular from 6 to 10 and specifically 7 carbon atoms. They may be present in linear, branched or cyclic form, preferably in linear or branched form. Examples are 2-pentanone (boiling point 102-105° C.), 3-pentanone, 2-hexanone, 3-hexanone, iso-butyl methyl ketone, 2-heptanone (boiling point 149-150° C.), 3-heptanone, cyclopentanone, cyclohexanone (boiling point 155° C.) or cycloheptanone. 2-Heptanone is preferred. Mixtures of the above aliphatic ketones are also possible. Usually, the emulsion according to the invention comprises from 1 to 50% by weight, preferably from 3 to 35% by weight, especially preferably from 5 to 25% by weight of aliphatic ketone, in each case based on the total weight of the emulsion.

Usually, suitable aromatic ketones comprise, in addition to an aromatic group, an aliphatic radical which is substituted by a ketone group. Suitable aromatic ketones are, for example, acetophenone or alkoxy-substituted acetophenone derivatives such as 4-methoxyacetophenone. Acetophenone is preferred. Usually, the emulsion according to the invention comprises from 0.5 to 50% by weight, preferably from 1 to 35% by weight, especially preferably from 2 to 25% by weight, of aromatic ketone, in each case based on the total weight of the emulsion.

The emulsion according to the invention comprises at least one surfactant (C). Surfactants are compounds which reduce the surface tension of water. Examples of surfactants are ionic (anionic or cationic) and nonionic surfactants. Usually, the emulsion according to the invention comprises at least one surfactant, i.e. one, two, three or more surfactants. It preferably comprises at least two surfactants. It especially preferably comprises at least three surfactants. In a preferred embodiment, it comprises two surfactants. In a very especially preferred embodiment, it comprises three surfactants.

Suitable ionic surfactants are the alkali, alkaline earth and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and of fatty acids, alkyl- and alkylarylsulfonates, alkylsulfates, lauryl ether sulfates and fatty alcohol sulfates, and salts of sulfated hexa-, hepta- and octadecanols, and of fatty alcohol glycol ethers, condensates of sulfonated naphthalene and its derivatives with formaldehyde, condensates of naphthalene or of the naphthalenesulfonic acids with phenol and formaldehyde, polycarboxylates (Sokalan® types, BASF, Germany) or phosphate esters of alkoxylated alcohols. Preferred ionic surfactants are anionic surfactants. Preferred anionic surfactants are phosphate esters of an alkoxylated alcohol. Especially suitable is a phosphate ester of an ethoxylated C₁₀₋₁₆-fatty alcohol with a degree of ethoxylation of from 3 to 15. In an especially preferred embodiment, the emulsion according to the invention comprises an ethoxylated fatty alcohol with a degree of ethoxylation of from 2 to 10; a poly(ethylene oxide block propylene oxide) or its derivatives with a mean molar mass of at least 2000 g/mol; and a phosphate ester of an alkoxylated alcohol.

Suitable nonionic surfactants are polyoxyethylene octyl phenol ethers, alkoxylated alcohols such as ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors and also proteins, denatured proteins, polysaccharides (for example methylcellulose), hydrophobically modified starches, polyvinyl alcohol (Mowiol® types, Clariant, Switzerland), polyalkoxylates, polyvinylamine (Lupamin® types, BASF, Germany), polyethyleneimine (Lupasol® types, BASF, Germany), polyvinylpyrrolidone and their copolymers or block polymers.

Preferred nonionic surfactants are alkoxylated alcohols and block polymers. The alkoxylated alcohol is preferably a fatty alcohol, in particular a fatty alcohol with 8 to 32, specifically with 9 to 18, carbon atoms in the fatty alcohol moiety which is alkoxylated with ethylene oxide (E0) or propylene oxide (PO). In most cases, the alkoxylated fatty alcohol has a degree of ethoxylation of from 1 to 30, preferably from 2 to 10 and specifically from 4 to 8 ethylene oxide groups and/or a degree of propoxylation of from 1 to 30, preferably from 2 to 15 and specifically from 3 to 10 propylene oxide groups. The block polymer is conventionally a di- or tri-block polymer or its derivative, with the polymeric moiety being composed of ethylene oxide and propylene oxide. The mean molar mass is usually at least 1000 g/mol, preferably at least 2000 g/mol. A substance which is specifically suitable is poly(ethylene oxide block propylene oxide) alkyl ether with a molar mass of at least 2000 g/mol and one C₁₋₁₀-alkylether unit. The nonionic surfactants are preferably an ethoxylated fatty alcohol with a degree of ethoxylation of from 2 to 10 and a poly(ethylene oxide block propylene oxide) or its derivatives with a mean molar mass of at least 2000 g/mol.

If the emulsion according to the invention comprises at least three surfactants, then the surfactant comprises a mixture of two nonionic and one anionic surfactant. Preferred is a mixture of alkoxylated alcohol, block polymer and phosphate ester of alkoxylated alcohols.

The emulsion according to the invention comprises from 2 to 50% by weight, preferably from 3 to 50% by weight, in particular from 5 to 50% by weight, specifically from 7 to 30% by weight and very specifically from 9 to 30% by weight of pesticide (D) based on the total weight of the emulsion.

In the present context, the term pesticide designates at least one active substance selected from the group of the fungicides, insecticides, nematocides, herbicides and/or safeners or growth regulators. Preferred pesticides are fungicides, insecticides and herbicides, in particular fungicides and insecticides. Mixtures of pesticides from two or more of the abovementioned classes may also be used. The skilled worker is familiar with those pesticides, which can be found for example in Pesticide Manual, 13th Ed. (2003), The British Crop Protection Council, London. The following list of pesticides identifies active substances which are possible, but is not intended to be limited thereto.

In a preferred embodiment, pesticides which are preferred are those which dissolve to at least 2% by weight, preferably to at least 5 and specifically to at least 10% by weight in solvent (A) at 20° C., in each case based on the weight of the solvent.

The fungicide is selected from among:

A) strobilurins:

azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, 2-(ortho(2,5-dimethylphenyloxymethylene)-phenyl)-3-methoxyacrylic acid methyl ester, 3-methoxy-2-(2-(N-(4-methoxyphenyl)-cyclopropanecarboximidoylsulfanylmethyl)phenyl)acrylic acid methyl ester, 2-(2-(3-(2,6-dichlorophenyl)-1-methylallylideneaminooxymethyl)phenyl)-2-methoxyimino-N-methylacetamide;

B) carboxamides:
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M, ofurace, oxadixyl, oxycarboxin, penthiopyrad, tecloftalam, thifluzamide, tiadinil, 2-amino-4-methylthiazole-5-carboxanilide, 2-chloro-N-(1,1,3-tri-methylindan-4-yl)nicotinamide, (2′,4′-difluorobiphenyl-2-yl) 3-difluoromethyl-1-methyl-1 H-pyrazole-4-carboxamide, (2′,4′-dichlorobiphenyl-2-yl) 3-difluoromethyl-1-methyl-1H-pyrazole-4-carboxamide, (2′,5′-difluorobiphenyl-2-yl) 3-difluoromethyl-1-methyl-1H-pyrazole-4-carboxamide, (2′,5′-dichlorobiphenyl-2-yl) 3-difluoromethyl-1-methyl-1H-pyrazole-4-carboxamide, (3′,5′-difluorobiphenyl-2-yl) 3-difluoromethyl-1-methyl-1H-pyrazole-4-carboxamide, (3′,5′-dichlorobiphenyl-2-yl) 3-difluoromethyl-1-methyl-1 H-pyrazole-4-carboxamide, (3′-fluorobiphenyl-2-yl) 3-difluoromethyl-1-methyl-1H-pyrazole-4-carboxamide, (3′-chlorobiphenyl-2-yl) 3-difluoromethyl-1-methyl-1H-pyrazole-4-carboxamide, (2′-fluorobiphenyl-2-yl) 3-difluoromethyl-1-methyl-1H-pyrazole-4-carboxamide, (2′-chlorobiphenyl-2-yl) 3-difluoromethyl-1-methyl-1H-pyrazole-4-carboxamide, (3′,4′,5′-trifluorobiphenyl-2-yl) 3-difluoromethyl-1-methyl-1H-pyrazole-4-carboxamide, (2′,4′,5′-trifluorobiphenyl-2-yl) 3-difluoromethyl-1-methyl-1H-pyrazole-4-carboxamide, [2-(1,1,2,3,3,3-hexafluoropropoxy)phenyl] 3-difluoromethyl-1-methyl-1H-pyrazole-4-carboxamide, [2-(1,1,2,2-tetrafluoroethoxy)phenyl] 3-difluoromethyl-1-methyl-1H-pyrazole-4-carboxamide, (4′-trifluoromethylthiobiphenyl-2-yl) 3-difluoromethyl-1-methyl-1H-pyrazole-4-carboxamide, N-(3′,4′-dichloro-5-fluorobiphenyl-2-yl)-3-difluoromethyl-1-methyl-1H-pyrazole-4-carboxamide, N-(2-(1,3-dimethylbutyl)phenyl)-1,3,3-trimethyl-5-fluoro-1H-pyrazole-4-carboxamide, N-(4′-chloro-3′,5′-difluorobiphenyl-2-yl)-3-difluoromethyl-1-methyl-1H-pyrazole-4-carboxamide, N-(4′-chloro-3′,5′-difluorobiphenyl-2-yl)-3-trifluoromethyl-1-methyl-1H-pyrazole-4-carboxamide, N-(3′,4′-dichloro-5′-fluorobiphenyl-2-yl)-3-trifluoromethyl-1-methyl-1H-pyrazole-4-carboxamide, N-(3′,5′-difluoro-4′-methylbiphenyl-2-yl)-3-difluoromethyl-1-methyl-1H-pyrazole-4-carboxamide, N-(3′,5′-difluoro-4′-methyl-biphenyl-2-yl)-3-trifluoromethyl-1-methyl-1H-pyrazole-4-carboxamide, N-(2-bicyclo-propyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide;
carboxylic acid morpholides: dimethomorph, flumorph;
benzamides: flumetover, fluopicolide, fluopyram, zoxamide, N-(3-ethyl-3,5,5-tri-methylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;

C) azoles:
triazoles: azaconazole, bitertanol, bromuconazole, cyproconazole, difenoconazole, diniconazole, diniconazole-M, epoxiconazole, fenbuconazole, fluquinconazole, flusilazol, flutriafol, hexaconazole, imibenconazole, ipconazole, metconazole, myclobutanil, oxpoconazole, paclobutrazol, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)cyclo-heptanol;
imidazoles: cyazofamid, imazalil, imazalil sulfate, pefurazoate, prochloraz, triflumizole;
benzimidazoles: benomyl, carbendazim, fuberidazole, thiabendazole;
others: ethaboxam, etridiazole, hymexazole, 2-(4-chlorophenyl)-N-[4-(3,4-dimethoxy-pheny)pisoxazol-5-yl]-2-prop-2-inyloxyacetamide;

D) nitrogen-containing heterocyclyl compounds
pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]pyridine, 2,3,5,6-tetra-chloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloro-pyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph acetate, fenpropimorph, tridemorph;
piperidines: fenpropidin;
dicarboximides: fluorimid, iprodione, procymidone, vinclozolin;
nonaromatic 5-ring heterocycles: famoxadon, fenamidon, octhilinone, probenazole, S-allyl 5-amino-2-isopropyl-3-oxo-4-ortho-tolyl-2,3-dihydropyrazole-1-thiocarboxylate;
others: acibenzolar-S-methyl, amisulbrom, anilazin, blasticidin-S, captafol, captan, quinomethionate, dazomet, debacarb, diclomezine, difenzoquat, difenzoquat methyl-sulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-di-methoxypyrimidin-2-yl)-2-methyl-1H-benzoimidazole, 5-chloro-7-(4-methylpiperidin-1-yl)-6-(2,4,6-trifluorophenyl)[1,2,4]triazolo[1,5-a]pyrimidine, 6-(3,4-dichlorophenyl)-5-methyl[1,2,4]triazolo[1,5-a]pyrimidin-7-ylamine, 6-(4-tert-butylphenyl)-5-methyl-[1,2,4]triazolo[1,5-a]pyrimidin-7-ylamine, 5-methyl-6-(3,5,5-trimethylhexyl)[1,2,4]-triazolo[1,5-a]pyrimidin-7-ylamine, 5-methyl-6-octyl[1,2,4]triazolo[1,5-a]pyrimidin-7-ylamine, 6-methyl-5-octyl[1,2,4]triazolo[1,5-a]pyrimidin-7-ylamine, 6-ethyl-5-octyl-[1,2,4]triazolo[1,5-a]pyrimidin-7-ylamine, 5-ethyl-6-octyl[1,2,4]triazolo[1,5-a]pyrimidin-7-ylamine, 5-ethyl-6-(3,5,5-trimethylhexyl)[1,2,4]triazolo[1,5-a]pyrimidin-7-ylamine, 6-octyl-5-propyl[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl[1,2,4]triazolo-[1,5-a]pyrimidin-7-ylamine and 5-trifluoromethyl-6-(3,5,5-trimethylhexyl)[1,2,4]-triazolo[1,5-a]pyrimidin-7-ylamine;

E) carbamates and dithiocarbamates
thio- and dithiocarbamates: ferbam, mancozeb, maneb, metam, methasulphocarb, metiram, propineb, thiram, zineb, ziram;
carbamates: diethofencarb, benthiavalicarb, iprovalicarb, propamocarb, propamocarb hydrochloride, valiphenal, N-(1-(1-(4-cyanophenyl)ethanesulfonyl)but-2-yl)carbamic acid 4-fluorophenyl ester;

F) other fungicides
guanidines: dodine, dodine free base, guazatine, guazatine acetate, iminoctadine, iminoctadine triacetate, iminoctadine tris(albesilate);
antibiotics: kasugamycin, kasugamycin hydrochloride hydrate, polyoxins, streptomycin, validamycin A;
nitrophenyl derivatives:

binapacryl, dicloran, dinobuton, dinocap, nitrothalisopropyl, tecnazene;
organometal compounds: fentin salts such as, for example, fentin acetate, fentin chloride, fentin hydroxide;
sulfur-containing heterocyclyl compounds: dithianon, isoprothiolane;
organophosphorus compounds: edifenphos, fosetyl, fosetyl-aluminum, iprobenfos, phosphorous acid and its salts, pyrazophos, tolclofosmethyl;
organochlorine compounds: chlorthalonil, dichlofluanid, dichlorphen, flusulfamide, hexachlorobenzene, pencycuron, pentachlorophenol and its salts, phthalide, quintozene, thiophanate-methyl, tolylfluanid, N-(4-chloro-2-nitrophenyl)-N-ethyl-4-methylbenzenesulfonamide;
inorganic active substances: phosphorous acid and its salts, Bordeaux mixture, copper salts such as, for example, copper acetate, copper hydroxide, copper oxychloride, basic copper sulfate, sulfur;
others: biphenyl, bronopol, cyflufenamid, cymoxanil, diphenylamin, metrafenon, mildiomycin, oxine-copper, prohexadione-calcium, spiroxamin, tolylfluanid, N-(cyclo-propylmethoxyimino(6-difluoromethoxy-2,3-difluorophenyl)methyl)-2-phenyl-acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methylformamidine.

Preferred fungicides are fungicides from the group of the strobilurins, such as azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, 2-(ortho(2,5-dimethylphenyloxymethylene)-phenyl)-3-methoxyacrylic acid methyl ester, 3-methoxy-2-(2-(N-(4-methoxyphenyl)-cyclopropanecarboximidoylsulfanylmethyl)phenyl)acrylic acid methyl ester, 2-(2-(3-(2,6-dichlorophenyl)-1-methylallylideneaminooxymethyl)phenyl)-2-methoxyimino-N-methyl-acetamide, and from the abovementioned groups of the morpholines, azoles and others.

Especially preferred fungicides are azoxystrobin, epoxiconazole, pyraclostrobin, kresoxim-methyl, carbendazim, metrafenone, boscalid, triticonazole, metconazole, fluquiconazole, dimethomorph, fenpropimorph, prochloraz, vinclozolin, iprodione, ditianon, metiram, tebuconazole, mancozeb, trifloxystrobin, chlorothalonil, metalaxyl, fosetyl, difemoconazole, cyprodinil, spiroxamine, prothioconazole or picoxystrobin. Particularly preferred are pyraclostrobin, fenpropimorph, metconazole and metrafenon.

The herbicide is selected from among:
acetamides: acetochlor, alachlor, butachlor, dimethachlor, dimethenamid, flufenacet, mefenacet, metolachlor, metazachlor, napropamid, naproanilid, pethoxamid, pretilachlor, propachlor, thenylchlor;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, tri-allate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazon, pyridate;
pyridines: aminopyralid, clopyralid, diflufenican, dithiopyr, fluridone, fluroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinon, meta-mitron, metribuzin, prometryn, simazine, terbuthylazine, terbutryn, triazifiam;
ureas: chiortoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalide, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsonic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrion, sulfentrazone, terbacil, tefuryltrione, tembotrione, thiencarbazone, topramezon, 4-hydroxy-3-[2-(2-methoxyethoxymethyl)-6-trifluoromethylpyridine-3-carbonyl]bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetic acid ethyl ester, 6-amino-5-chloro-2-cyclopropylpyrimidine-4-carboxylic acid methyl ester, 6-chloro-3-(2-cyclopropyl-6-methylphenoxy)pyridazin-4-ol, 4-amino-3-chloro-6-(4-chlorophenyl)-5-fluoro-pyridine-2-carboxylic acid, 4-amino-3-chloro-6-(4-chloro-2-fluoro-3-methoxyphenyl)-pyridine-2-carboxylic acid methyl ester, and 4-amino-3-chloro-6-(4-chloro-3-dimethyl-amino-2-fluorophenyl)pyridine-2-carboxylic acid methyl ester.

Preferred herbicides are those from the group of the acetamides, such as acetochlor, alachlor, butachlor, dimethachlor, dimethenamid, flufenacet, mefenacet, metolachlor, metazachlor, napropamid, naproanilid, pethoxamid, pretilachlor, propachlor, thenylchlor; from the group of the dinitroanilines, such as benfluralin, ethalfiuralin, oryzalin, pendimethalin, prodiamine, trifluralin; and from the group of the abovementioned others. Especially preferred herbicides are metazachlor, pendimethalin and topramezon.

The insecticide/nematicide is selected from among:
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyha-lothrin, permethrin, prallethrin, pyrethrin I and II, resmethrin, silafluofen, tau-fluva-linat, tefluthrin, tetramethrin, tralomethrin, transfluthrin, profluthrin, dimefluthrin,
insect growth inhibitors: a) chitin synthesis inhibitors: benzoylureas: chlorfluazuron, cyromazine, diflubenzuron, flucycloxuron, flufenoxuron, hexaflumuron, lufenuron, novaluron, teflubenuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl[1,3,5]triazinane;
GABA antagonists: endosulfan, ethiprole, fipronil, vaniliprole, pyrafluprole, pyriprole, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnon;
decouplers: chiorfenapyr;
inhibitors of oxidative phosphorylation: cyhexatin, diafenthiuron, fenbutatin oxide, propargite;
insect molting inhibitors: cyromazine;
mixed-function oxidase inhibitors: piperonyl butoxide;
sodium channel blockers: indoxacarb, metaflumizone;
others: benclothiaz, bifenazate, cartap, flonicamid, pyridalyl, pymetrozin, sulfur, thiocyclam, flubendiamid, chlorantraniliprole, cyazypyr (HGW86); cyenopyrafen, flupyrazofos, cyflumetofen, amidoflumet, imicyafos, bistrifluron, and pyrifluquinazon.

Preferred inseciticides are those from the group of the pyrethroids, such as allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin; from the group of the insect growth inhibitors, such as a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezine, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate; from the group of the GABA antagonists, such as endosulfan, ethiprole, fipronil, vaniliprol, pyrafluprole, pyriprole, 5-amino-1-(2,6-dichloro-4-methylphenyl)-4-sulfinamoyl-1H-pyrazole-3-thiocarboxamide; from the group of the nicotine receptor agonists/antagonists, such as clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl[1,3,5]triazinane; and from the group of the sodium channel blockers, such as indoxacarb, metaflumizone.

Especially preferred insecticides are fipronil, flufenoxuron, teflubenzuron, metaflumizone or alpha-cypermethrin. The insecticide alpha-cypermethrin is very especially preferred.

The growth regulator is selected from among:

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpyridine, ethephon, flumetralin, flurprimidol, fluthiacet, forchlorfenuron, gibberellic acid, inabenfid, indole-3-acetic acid, maleic hydrazide, mefluidid_{;} mepiquat (mepiquat chloride), metconazole, naphthaleneacetic acid, N-6-benzyladenine, paclobutrazole, prohexadione (prohexadione calcium), prohydrojasmone, thidiazuron, triapenthenol, tributyl phosphorotrithioate, 2,3,5-triiodo-benzoic acid, trinexapac-ethyl and uniconazole.

The oil-in-water emulsion according to the invention preferably comprises from 1 to 50% by weight of aromatic hydrocarbon, from 1 to 50% by weight of ketone, from 0.1 to 40% by weight of surfactant, from 2 to 50% by weight of pesticide and water to 100% by weight. The emulsion especially preferably comprises from 10 to 30% by weight of aromatic hydrocarbon, from 10 to 40% by weight of ketone, from 2 to 20% by weight of surfactant, from 7 to 30% by weight of pesticide, and water to 100% by weight.

In a preferred embodiment, the emulsion comprises from 10 to 30% by weight of aromatic hydrocarbon, from 1 to 20% by weight of ketone, from 2 to 20% by weight of surfactant, from 2 to 10% by weight of alpha-cypermethrin, and water to 100% by weight. In a very especially preferred embodiment, the emulsion comprises from 10 to 30% by weight of aromatic hydrocarbon, from 1 to 20% by weight of aliphatic ketone having 6 to 12 C atoms, from 1 to 20% by weight of acetophenone or its derivatives, from 2 to 20% by weight of surfactant, from 2 to 10% by weight of alpha-cypermethrin, and water to 100% by weight.

All the abovementioned embodiments of the oil-in-water emulsion according to the invention will hereinbelow be referred to as emulsion according to the invention.

The present invention also relates to a process for the preparation of an oil-in-water emulsion according to the invention by mixing solvent (A), water (B), surfactant (C) and at least 2% by weight of pesticide (D) based on the emulsion with one another. In a preferred embodiment, in the process for the preparation of an oil-in-water emulsion according to the invention, an oil phase comprising solvent (A) and pesticide (D) is dispersed with an aqueous phase. Preferably, the aqueous phase comprises at least one further adjuvant selected among antifreeze agent, bactericide or antifoam. The further adjuvant preferably comprises an antifreeze agent.

Usually, the process according to the invention comprises the mixing of an oil phase and an aqueous phase. To this end, the two phases may be provided separately. In most cases, the oil phase is prepared by dissolving a pesticide (D) in solvent. Surfactant (C) and other adjuvants may optionally be added. The aqueous phase can be prepared by adding water (B) and, optionally, surfactant (C) and further adjuvants. It is preferred to add the surfactants and further adjuvants to the phase in which they dissolve better. During the preparation, each of the two phases can be warmed and/or agitated mechanically. It is preferred that oil phase and aqueous phase are a homogenous, clear solution.

To prepare an oil-in-water emulsion, the two phases are conventionally mixed, with energy being supplied. Preferably, the two phases are dispersed by supplying energy. Mixing is preferably effected by supplying energy into the mixture of the phases by means of shaking, beating, stirring, turbulent mixing (for example rotor-stator mixer); by injecting one fluid into another; by oscillations and cavitation in the mixture (for example ultrasound); by means of emulsifying centrifuges; by means of colloid mills and homogenizers; or by means of a jet nozzle. Mixing usually takes place at from 5 to 90° C., preferably at from 10 to 50° C.

The present invention also relates to an agrochemical formulation comprising the emulsion according to the invention and further adjuvants. Suitable further adjuvants are the adjuvants conventionally used in agrochemical formulations, the choice of the adjuvants depending on the specific use form or the active substance. Examples of suitable adjuvants are additional solvents, surface-active substances (such as solubilizers, protective colloids, wetters and adhesives), organic and inorganic thickeners, bactericides, antifreeze agents, antifoams, if appropriate colorants and tackifiers (for example for the treatment of seed).

The agrochemical formulation according to the invention is prepared by mixing the emulsion according to the invention with the further adjuvants. All or individual further adjuvants may optionally already be added to the oil phase or the aqueous phase during the process for the preparation of an oil-in-water emulsion.

Additional solvents which are suitable and which may be present as adjuvant in addition to the abovementioned aromatic hydrocarbon and the ketone are organic solvents such as mineral oil fractions of medium to high boiling point such as kerosene and diesel oil, furthermore coal tar oils and oils of vegetable or animal origin, aliphatic, cyclic and aromatic hydrocarbons, for example paraffins, tetrahydronaphthalene, alkylated naphthalenes and their derivatives, alkylated benzenes and their derivatives, alcohols such as methanol, ethanol, propanol, butanol and cyclohexanol, glycols, ketones such as cyclohexanone, gamma-butyrolactone, dimethyl fatty acid amides, fatty acids and fatty acid esters, and strongly polar solvents, for example amines such as N-methylpyrrolidone. In principle, solvent mixtures may also be used. It is preferred to add to the emulsion according to the invention not more than 10% by weight of, preferably not more than 5% by weight of and especially preferably no, additional solvents.

In a further preferred embodiment, the solvent (A) comprises less than 10% by weight, preferably less than 5% by weight, especially preferably less than 2% by weight and in particular less than 0.5% by weight of solvent in addition to the abovementioned solvents (a), (b) and/or (c), in each case based on the total weight of the emulsion. In particular, the solvent (A) is free from lactones such as γ-butyrolactone.

Suitable surface-active substances (adjuvants, wetters, adhesives, dispersants or emulsifiers) in addition to the abovementioned surfactants are the alkali, alkaline earth and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and of fatty acids, alkyl- and alkylarylsulfonates, alkylsulfates, lauryl ether sulfates and fatty alcohol sulfates, and salts of sulfated hexa-, hepta- and octadecanols, and of fatty alcohol glycol ethers, condensates of sulfonated naphthalene and its derivatives with formaldehyde, condensates of naphthalene or of the naphthalenesulfonic acids with phenol and formaldehyde, polyoxyethylene octyl phenol ethers, ethoxylated isooctyl-, octyl- or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors and also proteins, denatured proteins, polysaccharides (for example methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and their copolymers.

The emulsion according to the invention may comprise large amounts of surface-active substances and surfactants. It may comprise from 0.1 to 40% by weight, preferably from 1 to 30 and in particular from 2 to 20% by weight total amount of surface-active substances and surfactants, based on the total amount of the emulsion.

Examples of thickeners (i.e. compounds which impart to the composition a modified flow behavior, i.e. high viscosity at rest and low viscosity in motion) are polysaccharides and organic and inorganic layer minerals such as xanthan gum (Kelzan®, CP Kelco, USA), Rhodopol® 23 (Rhodia, France) or Veegum® (R. T. Vanderbilt, USA) or Attaclay® (Engelhard Corp., N.J., USA).

Bactericides may be added to stabilize the composition. Examples of bactericides are those based on diclorophen and benzyl alcohol hemiformal (Proxel® from ICI or Acticide® RS from Thor Chemie and Kathon® MK from Rohm & Haas) and isothiazolinone derivatives such as alkylisothiazolinones and benzoisothiazolinones (Acticide® MBS from Thor Chemie).

Examples of suitable antifreeze agents are ethylene glycol, propylene glycol, urea and glycerol.

Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and their mixtures.

Examples of colorants are both pigments, which are sparingly soluble in water, and dyes, which are soluble in water. Examples which may be mentioned are the dyes and pigments known by the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of stickers are polyvinylpyrrolidone, polyvinyl acetate, polyvinyl alcohol and cellulose ethers (Tylose®, Shin-Etsu, Japan).

The present invention also relates to a method of controlling phytopathogenic fungi and/or undesired vegetation and/or undesired attack by insects or mites and/or for regulating the growth of plants, where an agrochemical formulation according to the invention is allowed to act on the respective pests, their environment or the plants to be protected from the respective pest, on the soil and/or on undesired plants and/or on the useful plants and/or their environment.

Furthermore, the invention relates to a method of controlling undesired attack by insects or mites on plants and/or of controlling phytopathogenic fungi and/or of controlling undesired vegetation, wherein seeds of useful plants are treated with an agrochemical formulation according to the invention.

In most cases, the agrochemical formulation is diluted with water prior to use (tank mix). The dilute agrochemical formulation is usually applied by spraying or atomizing.

It is possible to add to the agrochemical formulation various types of oils, or wetters, adjuvants, herbicides, bactericides, other fungicides and/or pesticides, if appropriate also immediately prior to application (tank mix). These agents can be added to the compositions according to the invention in the weight ratio of from 1:100 to 100:1, preferably from 1:10 to 10:1. Adjuvants for this purpose which are especially suitable are: organically modified polysiloxanes, for example Break Thru S 240®; alcohol alkoxylates, for example Atplus® 245, Atplus® MBA 1303, Plurafac® LF and Lutensol® ON ; EO/PO block polymers, for example Pluronic® RPE 2035 and Genapol® B; alcohol ethoxylates, for example Lutensol® XP 80; and sodium dioctyl sulfosuccinate, for example Leophen® RA.

The active substance concentrations in the ready-to-use preparations may be varied within substantial ranges. In general, they are between 0.0001 and 10%, preferably between 0.01 and 1%. When used in plant protection, the applications are between 0.01 and 2.0 kg of active substance per ha, depending on the nature of the desired effect.

The control of undesirable vegetation is understood to be the destruction of weeds. Weeds, in the broadest sense, are understood as being those plants which grow at locations at which they are not desired, such as, for example:

Dicotyledonous weeds of the genus: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds of the genus: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

Specifically, the agrochemical formulations according to the invention are suitable for controlling the following plant diseases:

Albugo spp. (white rust) in ornamentals, vegetable crops (for example: A. candida) and sunflowers (for example A. tragopogonis); Alternaria spp. (black spot, brown rot) in vegetables, oilseed rape (for example A. brassicola or A. brassicae), sugarbeet (for example A. tenuis), fruit, rice, soybeans and in potatoes (for example A. solani or A. alternata) and tomatoes (for example A. solani or A. alternata) and Alternaria spp. (leaf blight) on wheat; Aphanomyces spp. in sugarbeet and vegetables; Ascochyta spp. in cereals and vegetables, for example A. tritici (leaf spot) in wheat and A. hordei in barley; Bipolaris and Drechslera spp. (teleomorph: Cochliobolus spp.) in maize (for example D. maydis), cereals (for example B. sorokiniana: common root rot), rice (for example B. oryzae) and turf; Blumeria (formerly: Erysiphe) graminis (powdery mildew) in cereals (for example wheat or barley); Botryosphaeria spp. (black dead arm disease) in grapevines (for example B. obtusa); Botrytis cinerea (teleomorph: Botryotinia fuckeliana: gray mold) in soft fruit and pome fruit (strawberries, inter alia), vegetables (lettuce, carrots, celery and cabbage, inter alia), oilseed rape, flowers, grapevines, forestry crops and wheat (head rot); Bremia lactucae (downy mildew) in lettuce; Ceratocystis (syn. Ophiostoma) spp. (bluing fungus) in deciduous and coniferous woody species, for example C. ulmi (Dutch elm disease) in elms; Cercospora spp. (cercospora leaf spot) in maize, rice, sugarbeet (for example C. beticola), sugarcane, vegetable, coffee, soybeans (for example C. sojina or C. kikuchii) and rice; Cladosporium spp. in tomatoes (for example C. fulvum: leaf mold disease) and cereals, for example C. herbarum (black ear) in wheat; Claviceps purpurea (ergot) in cereals; Cochliobolus (anamorph: Helminthosporium or Bipolaris) spp. (leaf spot) in maize (for example C. carbonum), cereals (for example C. sativus, anamorph: B. sorokiniana, common root rot) and rice (for example C. miyabeanus, anamorph: H. oryzae); Colletotrichum (teleomorph: Glomerella) spp. (leaf spot, anthracnose) in cotton (for example C. gossypii), maize (for example C. graminicola: red stalk rot and leaf spot), soft fruit, potatoes (for example C. coccodes: foot rot), beans (for example C. lindemuthianum) and soybeans (for example C. truncatum); Corticium spp., for example C. sasakii (bordered sheath spot) in rice; Coynespora cassiicola (leaf spot) in soybeans and ornamentals; Cycloconium spp., for example C. oleaginum in olives; Cylindrocarpon spp. (for example fruit tree canker or black foot disease, teleomorph: Nectria or Neonectria spp.) in woody fruiting species, grapevines (for example C. liriodendri, teleomorph: Neonectria liriodendri, black foot disease) and many woody ornamentals; Dematophora (teleomorph: Rosellinia) necatrix (root/stem rot) in soybeans; Diaporthe spp., for example D. phaseolorum (soybean stem canker) in soybeans; Drechslera (syn. Helminthosporium, teleomorph: Pyrenophora) spp. in maize, cereals such as barley (for example D. teres, net blotch) and in wheat (for example D. tritici-repentis: DTR), rice and turf; esca disease (apoplexy) in grapevines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. in pome fruit (E. pyre) and soft fruit (E. veneta: cane spot) and grapevines (E. ampelina: grapevine anthracnose); Entyloma oryzae (leaf smut) in rice; Epicoccum spp. (black ear) in wheat; Erysiphe spp. (powdery mildew) in sugarbeet (E. betae), vegetables (for example E. pisi), such as cucurbits (for example E. cichoracearum) and brassicas such as oilseed rape (for example E. cruciferarum); Eutypa lata (grape canker or grape vine dieback, anamorph: Cytosporina lata, syn. Libertella blepharis) in woody fruiting trees, grapevines and many woody ornamentals; Exserohilum (syn. Helminthosporium) spp. in maize (for example E. turcicum); Fusarium (teleomorph: Gibberella) spp. (wilt, foot rot and culm rot) in various plants such as, for example F. graminearum or F. culmorum (foot rot, partial ear sterility) in cereals (for example wheat or barley), F. oxysporum in tomatoes, F. solani on soybeans and F. verticilliodes in maize; Gaeumannomyces graminis (take-all) in cereals (for example wheat or barley) and maize; Gibberella spp. in cereals (for example G. zeae) and rice (for example G. fujikuror: bakanae disease); Glomerella cingulata in grapevines, pome fruit and other plants and G. gossypiii cotton; grain staining complex in rice; Guignardia bidwellii (black rot) in grapevines; Gymnosporangium spp. in Rosaceae and juniper, for example G. sabinae (pear rust) in pears; Helminthosporium spp. (syn. Drechslera, teleomorph: Cochliobolus) in maize, cereals and rice; Hemileia spp., for example H. vastatrix (coffee leaf rust) in coffee; Isarlopsis clavispora (syn. Cladosporium vitis) in grapevines; Macrophomina phaseollna (syn. phaseoli) (root rot/charcoal rot) in soybeans and cotton; Microdochium (syn. Fusarium) nivale (snow mold) in cereals (for example wheat or barley); Microsphaera diffusa (powdery mildew) in soybeans; Monllinia spp., for example M. laxa, M. fructicola and M. fructigena (blossom blight and spur canker) in stone fruit and other Rosaceae; Mycosphaerella spp. in cereals, bananas, soft fruit and peanuts such as, for example, M. graminicola (anamorph: Septoria tritici, septoria leaf blotch) in wheat or M. fijiensis (black Sigatoka disease) in bananas; Peronospora spp. (downy mildew) in cabbage (for example P. brassicae), oilseed rape (for example P. parasitica), alliums (for example P. destructor), tobacco (P. tabacina) and soybeans (for example P. manshurica); Phakopsora pachyrhizi and P. meibomiae (soybean rust) in soybeans; Phialophora spp. for example in grapevines (for example P. tracheiphila and P. tetraspora) and soybeans (for example P. gregata: brown stem rot); Phoma lingam (phoma stem canker) in oilseed rape and cabbage and P. betae (leaf spot) in sugarbeet; Phomopsis spp. in sunflowers, grapevines (for example P. viticola: phomopsis cane and leaf spot) and soybeans (for example stem canker: P. phaseoli: teleomorph: Diaporthe phaseolorum); Physoderma maydis (brown spot) in maize; Phytophthora spp. (root, root rot, leaf rot, stem rot and fruit rot) in a variety of plants such as in bell pepper and cucurbits (for example P. capsici), soybeans (for example P. megasperma, syn. P. sojae), potatoes and tomatoes (for example P. infestans: late blight) and deciduous woody species (for example P. ramorum: sudden oak death); Plasmodiophora brassicae (clubroot disease) in cabbage, oilseed rape, radish and other plants; Plasmopara spp., for example P. viticola (downy mildew) in grapevines and P. halstedii sunflowers; Podosphaera spp. (powdery mildew) in Rosaceae, hops, pome fruit and soft fruit, for example P. leucotricha in apple; Polymyxa spp., for example in cereals such as barley and wheat (P. graminis) and sugarbeet (P. betae), and the virus diseases transmitted thereby; Pseudocercosporella herpotrichoides (eyespot, teleomorph: Tapesia yallundae) in cereals, for example wheat or barley; Pseudoperonospora (downy mildew) in a variety of plants, for example P. cubensis in cucurbits or P. humuli in hops; Pseudopezicula tracheiphlla (red fire disease, anamorph: Phialophora) in grapevines; Puccinia spp. (rust) in a variety of plants, for example P. triticina (leaf rust of wheat), P. striiformis (yellow rust), P. hordei (brown rust), P. graminis (stem rust) or P. recondita (brown leaf rust of rye) in cereals such as, for example, wheat, barley or rye, and in asparagus (for example P. asparagi); Pyrenophora (anamorph: Drechslera) tritici-repentis (tan spot) in wheat or P. teres (net blotch) in barley; Pyricularia spp., for example P. oryzae (teleomorph: Magnaporthe grisea, rice blast) in rice and P. grisea in turf and cereals; Pythium spp. (damping-off) in turf, rice, maize, wheat, cotton, oilseed rape, sunflowers, sugarbeet, vegetables and other plants (for example P. ultimum or P. aphanidermatum); Ramularia spp., for example R. collo-cygni (ramularia leaf spot/physiological leaf spots) in barley and R. beticola in sugarbeet; Rhizoctonia spp. in cotton, rice, potatoes, turf, maize, oilseed rape, sugarbeet, vegetables and a Variety of other plants, for example R. solani (rhizoctonia root/stem rot) in soybeans, R. solani (bordered sheath spot) in rice or R. cerealis (sharp eyespot) in wheat or barley; Rhizopus stolonifer (black bread mold) in strawberries, carrots, cabbage, grapevines and tomatoes; Rhynchosporium secalis (leaf spot) in barley, rye and triticale; Sarocladium oryzae and S. attenuatum (sheath rot) in rice; Sclerotinia spp. (stem rot, sclerotinia disease) in vegetable crops and arable crops such as oilseed rape, sunflowers (for example Sclerotinia sclerotiorum) and soybeans (for example S. rolfsii); Septoria spp. in a variety of plants, for example S. glycines (septoria blight) in soybeans, S. tritici (Septoria tritici blotch) in wheat and S. (syn. Stagonospora) nodorum (Stagonospora nodorum leaf and glume blotch) in cereals; Uncinula (syn. Erysiphe) necator (powdery mildew, anamorph: Oidium tucker) in grapevines; Setospaeria spp. (maize leaf blight) in maize (for example S. turcicum, syn. Helminthosporium turcicum) and turf; Sphacelotheca spp. in maize, (for example S. reiliana: head smut), millet/sorghum and sugarcane; Sphaerotheca fuliginea (powdery mildew) in cucurbits; Spongospora subterranea (powdery scab) in potatoes and the virus diseases transmitted thereby; Stagonospora spp. in cereals, for example S. nodorum (Stagonospora nodorum leaf and glume blotch, teleomorph: Leptosphaeria [syn. Phaeosphaeria] nodorum) in wheat; Synchytrium endobioticum in potatoes (potato wart disease); Taphrina spp., for example T. deformans (leaf curl) in peach and T. pruni (plum pocket disease) in plums; Thielaviopsis spp. (black root rot) in tobacco, pome fruit, vegetable crops, soybeans and cotton, for example T. basicola (syn. Chalara elegans); Tilletia spp. (stinking smut or smooth-spored bunt) in cereals such as, for example, T. tritici (syn. T. caries, wheat bunt) and T. controversa (dwarf bunt) in wheat; Typhula incarnata (snow blight) in barley or wheat; Urocystis spp., for example U. occulta (stalk smut) in rye; Uromyces spp. (rust) in vegetable plants such as beans (for example U. appendiculatus, syn. U. phaseoli) and sugarbeet (for example U. betae); Ustilago spp. (loose smut) in cereals (for example U. nuda and U. avaenae), maize (for example U. maydis: maize blister smut) and sugarcane; Venturia spp. (scab) in apples (for example V. inaequalis) and pears; and Verticillium spp. (verticillium wilt, tip rot) in a variety of plants such as woody fruiting and ornamental species, grapevines, soft fruit, vegetable crops and arable crops such as, for example, V. dahliae in strawberries, oilseed rape, potatoes and tomatoes.

The agrochemical formulations according to the invention are suitable for controlling harmful fungi in the protection of materials and buildings (for example timber, paper, dispersions for painting, fibers or wovens) and in the protection of stored products. Harmful fungi which are of particular importance in the protection of timber and buildings are: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and moreover, in the protection of materials, the following yeasts: Candida spp. and Saccharomyces cerevisae.

The invention furthermore relates to seed which has been treated with an agrochemical formulation according to the invention. In a preferred embodiment, the seed comprises the agrochemical formulation according to the invention. These agrochemical formulations can be applied to the seed in undiluted or, preferably, diluted form. In this context, the composition in question can be diluted by a factor of 2 to 10, so that from 0.01 to 60% by weight, preferably from 0.1 to 40% by weight of active substance are present in the compositions to be used for the seed treatment. Application can be effected prior to sowing. The treatment of plant propagation material, in particular the treatment of seed, is known to the skilled worker and is carried out by dusting, coating, pelleting, immersing, or soaking the plant propagation material, the treatment preferably being effected by pelleting, coating and dusting, so that for example premature germination of the seed is prevented. In the treatment of seed, amounts of active substance of from 1 to 1000 g/100 kg, preferably from 5 to 100 g/100 kg of propagation material or seed are generally used.

The advantages of the emulsion according to the invention are high storage stability over several months. In particular, no phase separation is observed during storage. Moreover, the emulsion according to the invention permits, if required, the addition of large amounts of fat-soluble or water-soluble adjuvants in the oil phase or the aqueous phase. The good storage stability is retained even when such substances are added. A further advantage is that the emulsion according to the invention has a higher biological activity than other liquid formulations, in particular emulsion concentrates (EC) or suspension concentrates (SC), of the same active substance with the same active substance concentration. Another advantage of the emulsion according to the invention over the EC and SC formulations is that fewer organic solvents are required since the latter can in part be replaced by the aqueous phase.

The high water content, or low solvent content, of EW formulations, in particular in comparison with EC formulations, is ecologically advantageous. Moreover, water-based EW formulations can be repackaged more readily, and their handling is less risky for the distributor and the farmer since the flashpoint is markedly lower.

The examples which follow illustrate the invention without imposing any limitation.

### EXAMPLES

Solvesso® 200 ND: aromatic hydrocarbon mixture, initial boiling point at 235° C., ending at 305° C., solidification point at approx. −16° C., naphthalene content approx. 0.3% by weight, commercially available from Exxon Mobil.

Genapol® X 060: ethoxylated isotridecyl alcohol, degree of ethoxylation approximately 6, commercially available from Clariant.

Rhodafac® RS 610: phosphate ester of ethoxylated branched tridecanol; degree of ethoxylation approximately 6, acid number 75-85.

Ethylan® NS 500 K: polyalkylene glycol butyl ether, the polyalkylene glycol unit being an EO/PO block polymer, OH number 17-26 mg KOH/g, HLB value 14, molar weight 5200 g/mol, commercially available from Akzo Nobel.

Lutensol® ON 30: ethoxylated short-chain saturated synthetic alcohol, degree of ethoxylation approximately 3, OH number approximately 190 mg KOH/g, HLB value approximately 9, commercially available from BASF SE.

Alkoxylate A: nonionic C₁₆₋₁₈-fatty alcohol alkoxylated with ethylene oxide and propylene oxide (PO), surface tension approximately 32 mN/m (as specified in DIN 53914, 1 g/l distilled water, 20° C.).

Alkoxylate B: nonionic C₁₃-alcohol alkoxylated with ethylene oxide and propylene oxide (PO), surface tension approximately 27-28 mN/m (as specified in EN 14370, 1 g/l distilled water, room temperature).

Lutensol® XP 80: ethoxylated C10-Guerbet alcohol, degree of ethoxylation approximately 8, OH number approximately 110 mg KOH/g, HLB value approximately 14, commercially available from BASF SE.

Atplus® 245: alcohol alkoxylate, HLB value approximately 14, acid number 1.0 mg KOH/g, liquid at 25° C., commercially available from Croda.

Alkoxylate C: nonionic C₉₋₁₁-alcohol alkoxylated with ethylene oxide and propylene oxide (PO), surface tension approximately 29-30 mN/m (as specified in EN 14370, 1 g/l distilled water, room temperature).

Luviskol® VA 64: poly(vinylpyrrolidone co-vinyl acetate), the monomers VP/VAc being present in the weight ratio 6/4.

Rhodorsil® 416: silicone-containing antifoam, commercially available from Rhodia.

Rhodorsil® 432: silicone-containing antifoam, active substance content in aqueous emulsion 30% by weight, commercially available from Rhodia.

Acticide® MBS: biocide based on 2-methyl-4-isothiazolin-3-one (MIT) and 1,2-benzo-isothiazolin-3-one (BIT), commercially available from Thor.

### Example 1A to 1L

### Storage Stability

The emulsions 1A to 1L were prepared as specified in the formulas in Table 1 as follows (data in % by weight):

First, the oil phase was prepared by dissolving pyraclostrobin and metconazole in a solvent mixture consisting of acetophenone, Solvesso 200 ND and 2-heptanone. Then, the solution was treated with emulsifiers (Genapol X 060, Rhodafac RS 610, Rhodafac RS 710-E and/or Witconol NS 500 K) and oil-soluble adjuvants (Lutensol ON 30, alkoxylate A, alkoxylate B and/or Lutensol XP 80).

The aqueous phase was prepared in a second container. To this end, antifreeze glycerol, biocide (Acticid MBS), protective colloid, antifoam (Rhodorsil 416 and/or Rhodorsil 432), water-soluble adjuvants (Atplus 245 and/or alkoxylate C) and demineralized water were admixed.

The aqueous phase was then stirred in a Silverson homogenizer at 4000 rpm. Then, the oil phase was added at room temperature to the sheared aqueous phase, and dispersing was continued for 15 minutes. This gives a yellowish to white oil-in-water emulsion with a mean particle size distribution (d₅₀) of approximately from 0.7 to 1.0 μm (determined using a Malvern Mastersizer 2000).

The emulsions 1A to 1L were stored for three months at 20° C. without agitating. All emulsions were stable, and no phase separation was observed.

### Example 2

### Biological Activity

The curative activity was tested on wheat against Puccinia recondita. To this end, the EW emulsions 1I to 1L of Example 1 were diluted with water so that a total active substance concentration of from 37.5, 75 and 150 ppm was obtained (Table 2, formulations EW-L to EW-O). In each case three pots with infected plants were then sprayed at an application rate of 200 I/ha. The infection with pustules, which were the result of Puccinia recondita, was determined after 7 days (score value). As a control, the infected plants were not treated, and the score values were also determined after 7 days (variant 0). It can be seen clearly that the biological efficacy of the EW formulations is very good, in particular at low active substance concentrations.

TABLE 2

| | | Active substance | Score value of | | | |
|---|---|---|---|---|---|---|
| | | concentration | pustules | | | |
| Variant | Formulation | [ppm] | Pot 1 | Pot 2 | Pot 3 | ø |
| 0 | Control | — | 90 | 90 | 90 | 90 |
| 7 | EW - I | 150 | 0 | 0 | 0 | 0 |
| 8 | | 75 | 0 | 0 | 0 | 0 |
| 9 | | 37.5 | 7 | 10 | 20 | 12 |
| 10 | EW - J | 150 | 0 | 0 | 0 | 0 |
| 11 | | 75 | 3 | 1 | 1 | 2 |
| 12 | | 37.5 | 5 | 10 | 10 | 8 |
| 13 | EW - K | 150 | 0 | 0 | 0 | 0 |
| 14 | | 75 | 3 | 3 | 3 | 3 |
| 15 | | 37.5 | 20 | 20 | 10 | 17 |
| 16 | EW - L | 150 | 0 | 0 | 0 | 0 |
| 17 | | 75 | 5 | 1 | 1 | 2 |
| 18 | | 37.5 | 10 | 7 | 15 | 11 |

### Example 3

### Storage Stability

The emulsions 3A and 3B were prepared in accordance with the formulas in Table 3, as follows (data in % by weight):

First, the oil phase was prepared by dissolving alpha-cypermethrin in a solvent mixture consisting of acetophenone, Solvesso 200 ND and 2-heptanone. Then, the solution was treated with emulsifiers (Genapol X 060, Rhodafac RS 610, Witconol NS 500 K). The aqueous phase was prepared in a second container. To this end, antifreeze glycerol, biocide (Acticid MBS), protective colloid, antifoam (Rhodorsil 416, Rhodorsil 432) and demineralized water were admixed.

The aqueous phase was then stirred in a Silverson homogenizer at 4000 rpm. Then, the oil phase was added at room temperature to the sheared aqueous phase, and dispersing was continued for 15 minutes. This gives a yellowish to white oil-in-water emulsion with a mean particle size distribution (d₅₀) of approximately from 0.7 to 1.0 μm (determined using a Malvern Mastersizer 2000 by laser diffraction on particles).

The emulsions 3A and 3B were stored for three months at 20° C. without agitating. All emulsions were stable, and no phase separation was observed.

TABLE 3
(all numerical values in % by weight)

| | A | B |
|---|---|---|
| Alpha-cypermethrin | 5.5 | 5.2 |
| Acetophenone | 2.9 | 3.1 |
| Solvesso 200 ND | 21.8 | 21.5 |
| 2-Heptanone | 8.7 | 8.7 |
| Genapol X 060 | 1.9 | 4.6 |
| Rhodafac RS 610 | 1.1 | 1.0 |
| Ethylan NS 500 K | 1.9 | 3.6 |
| Glycerol | 11.4 | 10.3 |
| Luviskol VA 64 | 1.6 | 1.5 |
| Rhodorsil 416 | 0.02 | 0.02 |
| Rhodorsil 432 | 0.02 | 0.02 |
| Acticide MBS | 0.22 | 0.21 |
| Water | 42.7 | 40.2 |
| Total | 100.0 | 100.0 |

TABLE 1
(all numerical information in % by weight)

| | | A | B | C | D | E | F | G | H | I | J | K | L |
|---|---|---|---|---|---|---|---|---|---|---|---|---|---|
| 1 | Pyraclostrobin | 6.3 | 6.3 | 6.3 | 6.3 | 6.3 | 6.3 | 6.3 | 6.3 | 6.3 | 6.3 | 6.3 | 9.5 |
| 2 | Metconazole | 3.9 | 3.9 | 3.9 | 3.9 | 3.9 | 3.9 | 3.9 | 3.9 | 3.9 | 3.9 | 3.9 | 5.8 |
| 3 | Acetophenone | 11.0 | 11.1 | 11.1 | 11.1 | 11.1 | 11.1 | 11.1 | 11.1 | 11.1 | 11.1 | 11.1 | 11.1 |
| 4 | Solvesso 200 ND | 12.1 | 12.2 | 12.2 | 12.2 | 12.2 | 12.2 | 12.2 | 12.2 | 12.2 | 12.2 | 12.2 | 12.2 |
| 5 | 2-Heptanone | 11.0 | 11.1 | 11.1 | 11.1 | 11.1 | 11.1 | 11.1 | 11.1 | 11.1 | 11.1 | 11.1 | 11.1 |
| 6 | Genapol X 060 | 1.9 | 1.9 | 1.9 | 1.9 | 1.9 | 9.7 | 1.9 | 1.9 | 1.9 | 1.9 | 1.9 | 1.9 |
| 7 | Rhodafac RS 610 | 1.0 | 1.0 | 1.0 | 1.0 | 1.0 | 1.0 | 1.0 | 1.0 | 1.0 | 1.0 | 1.0 | 1.0 |
| 8 | Witconol NS 500 K | 1.5 | 1.5 | 1.5 | 1.5 | 1.5 | 1.5 | 1.5 | 1.5 | 1.5 | 1.5 | 1.5 | 1.5 |
| 9 | Lutensol ON 30 | | | | | 9.7 | | | | | 14.6 | 4.9 | 9.7 |
| 10 | Alkoxylate A | | | 9.7 | | | | | | | | | |
| 11 | Alkoxylate A/ | | 9.7 | | | | | | | | | | |
| | Solvesso 200ND 60:40 | | | | | | | | | | | | |
| 12 | Alkoxylate B | | | | 9.7 | | | | | 14.6 | | | |
| 13 | Lutensol XP 80 | | | | | | | | | | | | |
| 14 | Atplus 245 | | | | | | | 9.7 | | | | 4.9 | |
| 15 | Alkoxylate C | | | | | | | | 9.7 | | | | |
| 16 | Glycerol | 9.7 | 9.7 | 9.7 | 9.7 | 9.7 | 9.7 | 9.7 | 9.7 | 9.7 | 9.7 | 9.7 | 9.7 |
| 17 | Luviskol VA 64 | 1.5 | 1.5 | 1.5 | 1.5 | 1.5 | 1.5 | 1.5 | 1.5 | 1.5 | 1.5 | 1.5 | 1.5 |
| 18 | Rhodorsil 416 | 0.02 | 0.02 | 0.02 | 0.02 | 0.02 | 0.02 | 0.02 | 0.02 | 0.02 | 0.02 | 0.02 | 0.02 |
| 19 | Rhodorsil 432 | 0.02 | 0.02 | 0.02 | 0.02 | 0.02 | 0.02 | 0.02 | 0.02 | 0.02 | 0.02 | 0.02 | 0.02 |
| 20 | Acticide MBS | 0.19 | 0.19 | 0.19 | 0.19 | 0.19 | 0.19 | 0.19 | 0.19 | 0.19 | 0.19 | 0.19 | 0.19 |
| 21 | Water | 39.8 | 30.0 | 30.0 | 30.0 | 30.0 | 32.0 | 30.0 | 30.0 | 25.1 | 25.1 | 30.0 | 24.9 |
| 22 | Total | 100.0 | 100.0 | 100.0 | 100.0 | 100.0 | 100.0 | 100.0 | 100.0 | 100.0 | 100.0 | 100.0 | 100.0 |

1-12. (canceled)

13. An oil-in-water emulsion comprising solvent (A), water (B), surfactant (C) and at least 2% by weight of pesticide (D) based on the emulsion, wherein the solvent comprises an aromatic hydrocarbon (a) and a ketone (b) and a further ketone (c) wherein the ketone (b) is an aliphatic ketone, and the ketone (c) is an aromatic ketone.

14. The emulsion according to claim 1, wherein the aromatic hydrocarbon (a) has a boiling point of at least 160° C., the ketone (b) is an aliphatic ketone having 6 to 12 C atoms and the ketone (c) comprises acetophenone or its derivatives.

15. The emulsion according to claim 13, wherein the aromatic hydrocarbon (a) is an aromatic hydrocarbon mixture with a boiling point of at least 160° C., the ketone (b) is 2-heptanone and the ketone (c) is acetophenone.

16. The emulsion according to claim 13, wherein the surfactant (C) comprises a mixture of two nonionic and one anionic surfactant.

17. The emulsion according to claim 13, wherein the emulsion has a mean droplet size in the range of from 0.1 to 1.2 μm.

18. The emulsion according to claim 13, wherein the solvent (A) comprises less than 2% by weight of solvent in addition to the abovementioned solvents (a), (b) and/or (c) based on the total weight of the emulsion.

19. The emulsion according to any of claims 1 to 6, wherein the emulsion comprises from 5 to 50% by weight of pesticide (D).

20. The emulsion according to claim 13, wherein the emulsion comprises from 5 to 40% by weight of aromatic hydrocarbon (a).

21. The emulsion according to claim 13, wherein the emulsion comprises from 3 to 35% by weight of aliphatic ketone (b).

22. The emulsion according to claim 13, wherein the emulsion comprises from 2 to 25% by weight of aromatic ketone (c).

23. A process for the preparation of an oil-in-water emulsion according to claim 13, comprising mixing solvent (A), water (B), surfactant (C) and at least 2% by weight of pesticide (D) based on the emulsion with one another.

24. The method according to claim 11, wherein an oil phase comprising solvent (A) and pesticide (D) is dispersed with an aqueous phase, wherein the aqueous phase comprises at least one further adjuvant selected from the group consisting of antifreeze agent, bactericide and antifoam.

25. An agrochemical formulation comprising the emulsion according to claim 13 and further adjuvants.

26. A method of controlling phytopathogenic fungi and/or undesired vegetation and/or undesired attack by insects or mites and/or for regulating the growth of plants, wherein an agrochemical formulation according to claim 25 is allowed to act on the respective pests, their environment or the plants to be protected from the respective pest, on the soil and/or on undesirable plants and/or on the useful plants and/or their environment.

27. A method of controlling undesired attack by insects or mites on plants and/or of controlling phytopathogenic fungi and/or of controlling undesired vegetation, wherein seeds of useful plants are treated with an agrochemical formulation according to claim 25.

28. Seed treated with an agrochemical formulation according to claim 25.

29. The method according to claim 27, wherein the aromatic hydrocarbon (a) has a boiling point of at least 160° C., the ketone (b) is an aliphatic ketone having 6 to 12 C atoms and the ketone (c) comprises acetophenone or its derivatives.

30. The method according to claim 27, wherein the aromatic hydrocarbon (a) is an aromatic hydrocarbon mixture with a boiling point of at least 160° C., the ketone (b) is 2-heptanone and the ketone (c) is acetophenone.

31. The method according to claim 27, wherein the surfactant (C) comprises a mixture of two nonionic and one anionic surfactant.

32. The method according to claim 27, wherein the emulsion has a mean droplet size in the range of from 0.1 to 1.2 μm.
